(19) United States
(12) Patent Application Publication
(10) Pub. No.: US 2009/0171414 A1
(43) Pub. Date: Jul. 2, 2009

(54) INTERROGATION OF STORED DATA IN IMPLANTABLE DEVICES

(75) Inventors: Jonathan H. Kelly, Woodbury, MN (US); James Kalgren, Lino Lakes, MN (US)

Correspondence Address: SCHWEGMAN, LUNDBERG & WOESSNER, P.A., P.O. BOX 2938, MINNEAPOLIS, MN, 55402, US

(73) Assignee: Cardiac Pacemakers, Inc., st paul, CA (US)

(21) Appl. No.: 12/260,506

(22) Filed: Oct. 29, 2008

Related U.S. Application Data
(60) Provisional application No. 61/017,689, filed on Dec. 30, 2007.

(51) Int. Cl. A61N 1/362; A61N 1/36

(52) U.S. Cl. 607/32; 607/60

(57) ABSTRACT

A system and method is provided for receiving user input to perform an initial interrogation of an implantable medical device (IMD) using an external device. The initial interrogation commences a communication session between the IMD and the external device. During the initial interrogation, first data is transferred from the IMD to the external device and displayed at the external device. The first data comprises device configuration data of the IMD. During the initial interrogation, background transfer of second data from the IMD to the external device is initiated during or after the display of first data, without requiring a user request for the second data. The second data comprises historical physiological episode data.

### CROSS-REFERENCE TO RELATED APPLICATION

This application claims the benefit of U.S. Provisional Application No. 61/017,689, filed on Dec. 30, 2007, under 35 U.S.C. §119(e), which is hereby incorporated by reference.

### TECHNICAL FIELD

This document pertains generally to implantable devices, and more particularly, but not by way of limitation, to interrogating an implantable device and rapidly retrieving stored data from the implantable device.

### BACKGROUND

Implantable medical devices (IMDs) are designed to be implanted into patients for various purposes such as heart function management and stimulation. Some examples of these devices include cardiac function management (CFM) devices such as implantable pacemakers, implantable cardioverter defibrillators (ICDs), cardiac resynchronization devices, and devices that include a combination of such capabilities. ICDs, for example, monitor for certain cardiac arrhythmias, such as ventricular fibrillation, and ventricular tachycardia, and can administer therapy in response to detection of an irregular event. In addition to monitoring for conditions and delivering therapy, modem ICDs can store a number of types of data that may be retrieved later by a caregiver.

Typically, IMDs communicate wirelessly with an external device. Examples of the external device can include an IMD programmer that provides bi-directional communication between the IMD and the caregiver, a patient activator that allows a patient to activate an implantable device, or a patient data display that reads and displays information from the IMD.

### OVERVIEW

In certain examples, an IMD can store “episode” information, which can include physiological or device data associated with a particular episode of a cardiac arrhythmia. The present inventors have recognized, among other things, that retrieval or display of episode information stored in an MD can be a time-consuming process due to the amount of the information being transferred. Consequently, the caregiver may be burdened by having to wait a significant amount of time for the episode information from the IMD to be transferred to an external device such as the IMD programmer.

Accordingly, a system and method is provided for receiving user input to perform an initial interrogation of an implantable medical device (IMD) using an external device. The initial interrogation commences a communication session between the IMD and the external device. During the initial interrogation, first data is transferred from the IMD to the external device. The first data comprises device configuration data of the IMD. During or completion of initial interrogation, background transfer is initiated of second data from the IMD to the external device, without requiring a user request for the second data. The second data comprises historical physiological episode data.

Example 1 describes a system. In this example, the system comprises an implantable medical device (IMD). In this example, the system also comprises an external device in communication with the IMD and configured to receive an user input to perform an initial interrogation of the IMD using the external device, initiate transfer of first data from the IMD to the external device during the initial interrogation, and automatically initiate background transfer of second data from the IMD to the external device during or after display of first data at the external device and without requiring a user request for the second data during the initial interrogation.

In Example 2, the system of Example 1 optionally comprises the external device being configured such that the background transfer of second data from the IMD to the external device begins before completing transfer of the first data from the IMD to the external device.

In Example 3, the system of one or more of Examples 1-2 is optionally configured such that background transfer of second data from the IMD to the external device begins after completing transfer of the first data from the IMD to the external device.

In Example 4, the system of one or more of Examples 1-3 is optionally configured such that the first data comprises at least one of device calibration data, device configuration data, device firmware data, device mode data, fault information data, magnet data, battery status data, impedance measurement data, encrypted patient data, trended data, atrial counter data, ventricular counter data, paced episode data or IMD date or time information.

In Example 5, the system of one or more of Examples 1-4 is optionally configured such that the second data comprises a stored detail episode data, the stored detailed episode data including historical physiological episode data corresponding to a particular episode.

In Example 6, the system of one or more of Examples 1-5 is optionally configured to receive a user input requesting specific historical physiological episode data from the IMD and in response to the user input requesting specific historical physiological episode data, to interrupt the automatically initiated background transfer of the second data.

In Example 7, the system of one or more of Examples 1-6 is optionally configured to preempt the automatically initiated background transfer of the second data from the IMD, and retrieve the specific historical physiological episode data from the IMD in response to the user input requesting the specific historical physiological episode data.

In Example 8, the system of one or more of Examples 1-7 is optionally configured to resume the automatically initiated background transfer of the second data from the IMD after retrieving the specific historical physiological episode data from the IMD.

In Example 9, the system of one or more of Examples 1-8 is optionally configured to initiate transfer of first data from the IMD to the external device, the first data including at least one of device calibration data, device configuration data, device firmware data, device mode data, fault information data, magnet data, battery status data, episode identification data, impedance measurement data, encrypted patient data, trended data, atrial counter data, ventricular counter data, paced episode data or IMD date or time information.

In Example 10, the system of one or more of Examples 1-9 is optionally configured to automatically initiate background transfer of second data including at least one of episode identification data, episode type data, episode detection data, therapy data, electrogram data or device marker data.

In Example 11, the system of one or more of Examples 1-10 is optionally configured to transfer the first data including a plurality of operational parameter values used to control operation of the implantable medical device.

In Example 12, the system of one or more of Examples 1-2 is optionally configured to perform the background transfer of the second data to transfer the most recent historical physiological episode data in the implantable medical device followed then by background transfer of preceding historical physiological episode data.

Example 13 describes a method comprising: receiving user input to perform an initial interrogation of an implantable medical device (IMD) using an external device, the initial interrogation commencing a communication session between the IMD and the external device; during the initial interrogation, initiating transferring of first data from the IMD to the external device, wherein the first data comprises device configuration data of the IMD; and during the initial interrogation, automatically initiating background transferring of second data from the IMD to the external device during or after display of first data at the external device, without requiring a user request for the second data, wherein the second data comprises historical physiological episode data.

In Example 14, the method of Example 13 is optionally performed such that the background transferring of second data includes beginning transfer of second data from the IMD to the external device before completing transfer of the first data from the IMD to the external device.

In Example 15, the method of one or more of Examples 13-14 is optionally performed such that the background transferring of second data includes beginning transfer of second data from the IMD to the external device after completing transfer of the first data from the IMD to the external device.

In Example 16, the method of one or more of Examples 13-15 is optionally performed such that the receiving, at the external device, user input requesting specific historical physiological episode data from the IMD; and in response to the user input requesting specific historical physiological episode data, interrupting the automatically initiated background transfer of the second data from the IMD, and retrieving the specific historical physiological episode data from the IMD.

In Example 17, the method of one or more of Examples 13-16 optionally comprises resuming the automatically initiated background transferring of the second data after retrieving the specific historical physiological episode data from the IMD.

In Example 18, the method of one or more of Examples 13-17 is optionally performed such that the transferring of first data includes transferring of first data including a plurality of operational parameter values used to control operation of the implantable medical device.

In Example 19, the method of one or more of Examples 13-18 is optionally performed such that the transferring of first data includes transferring at least one of: device calibration data, device configuration data, device firmware data, device mode data, fault information data, magnet data, battery status data, impedance measurement data, encrypted patient data, trended data, atrial counter data, ventricular counter data, paced episode data or IMD date or time information.

In Example 20, the method of one or more of Examples 13-19 is optionally performed such that the background transferring of second data comprises background transferring of stored detail episode information including at least one of episode identification data, episode type data, episode detection data, therapy data, electrogram data and device marker data.

In Example 21, the method of Example 13-20 is optionally performed such that the background transferring of second data comprises first background transferring the most recent historical physiological episode data in the implantable medical device followed then by background transferring preceding historical physiological episode data.

Example 22 describes a computer readable medium encoded with instructions. In this Example, the instructions when executed comprises: receiving user input to perform an initial interrogation of the IMD using an external device, the initial interrogation commencing a communication session between the IMD and the external device; the initial interrogation, initiating transfer of first data from the IMD to the external device; and during or after the initial interrogation, automatically initiating background transfer of second data from the IMD to the external device during display of first data at the external device, without requiring a user request for the historical physiological episode data, wherein the second data comprises historical physiological episode data.

In Example 23, the instructions of Example 22 when executed comprises beginning transfer of second data from the IMD to the external device before completing transfer of the first data from the IMD to the external device.

In Example 24, the instructions of Example 22 when executed comprises beginning transfer of second data from the IMD to the external device after completing transfer of the first data from the IMD to the external device.

In Example 25, the instructions of Example 22 when executed comprises receiving, at the external device, user input requesting specific historical physiological episode data from the IMD; and in response to the user input requesting specific historical physiological episode data, preempting the automatically initiated background transferring of the second data comprising historical physiological episode data from the IMD, and retrieving the specific historical physiological episode data from the IMD.

In Example 26, the instructions of Example 22 when executed comprises resuming the automatically initiated background transferring of the second data after retrieving the specific historical physiological episode data from the IMD.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A is an illustration of an example of an implantable device and an external device.

FIG. 1B is an illustration of an example of a CRM system, including an implantable medical device and an external system, and portions of an environment in which it is used.

FIG. 2 is a block diagram illustrating an example of at least one kind of stored data that can be transferred from the IMD to the external device during the interrogation of implantable medical devices.

FIG. 3 illustrates an example of transferring stored data during interrogation of implantable medical devices.

FIG. 4 is a block diagram illustrating an example of an external device, such as a computer system comprising instructions for performing one or more of the techniques described herein.

### DETAILED DESCRIPTION

An implantable medical device (IMD) generally refers to any medical device that can be implanted in a patient. By way of example, but not limitation, an IMD can be operable to sense a physiologic parameter, such as blood pressure, temperature, posture, blood sugar levels, or others. Some IMDs, such as implantable cardioverter-defibrillators (ICDs) and pacemakers (PMs), can sense and store electrogram (EGM) data. An IMD can be operable to provide therapy, such as, but not limited to, delivering electrical pulses for rhythm management or resynchronization in a patient's heart. An IMD may provide other functions, such as communication. In certain examples, an IMD can transmit stored data to an external device, such as an IMD programmer recorder/monitor (PRM) or in-home monitoring device.

The present inventors have recognized, among other things, an efficient strategy for transfer of stored data during interrogation of implantable medical devices.

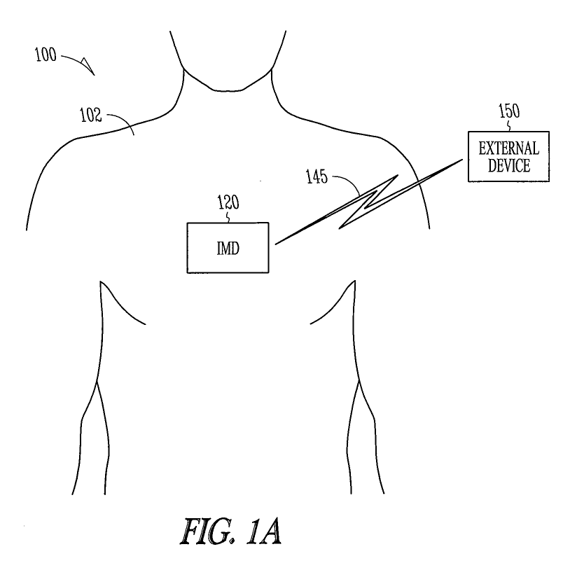

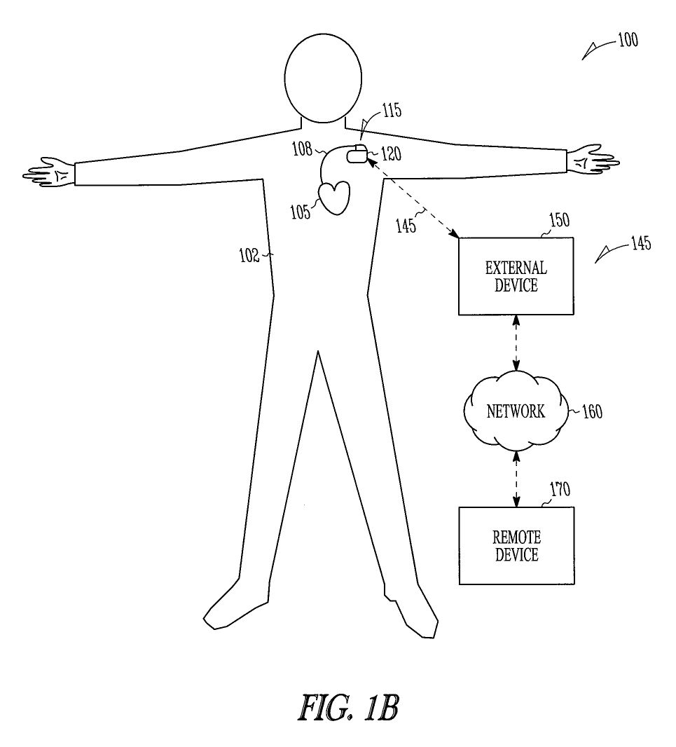

FIG. 1A is an illustration of an example of a system 100 including an implantable medical device (IMD) 120 that can be implanted in a body 102, and that can be in communication with an external device 150. In an example, the external device 150 can be configured to receive device data from the IMD 120. In certain examples, the device data can include parameter values corresponding to various device parameters. In certain examples, the external device 150 can also be configured to receive from the IMD 120 episode information that may be transmitted by the implantable medical device 120 over a telemetry link 145. In an example, telemetry link 145 can include an inductive telemetry link. In another example, telemetry link 145 can include a far-field radio-frequency telemetry link.

In an example, various device parameters transmitted by the implantable medical device 120 during initial interrogation can include various values related to calibration, algorithm, device configuration, etc. In an example, various device parameters transferred from implantable medical device 120 to external device 150 during initial interrogation can include various counters that can count occurrences of patient events, such as related to atrial tachycardia, ventricular tachycardia, atrial bradycardia, ventricular bradycardia, etc. In an example, the device data transferred during initial interrogation can include histograms that are related to atrial arrhythmia, ventricular arrhythmia, atrial pacing, ventricular pacing, etc. In an example, the device parameter data transferred from the IMD 120 to the external device 150 during initial interrogation can include trended physiological data, such as can be related to respiratory rate, patient activity, heart rate variability, etc. In an example, the device parameter data transferred during initial interrogation can include fault information data, such as related to any faults detected in the IMD 120. In an example, the device parameter data transferred during initial interrogation can include magnet data parameters which can configure the device behavior upon application of a magnetic field on the IMD. In some examples, magnet data parameter can be configured to initiate or withhold therapy when a magnetic field is applied on the IMD.

In an example, the device data communicated from the IMD 120 during the initial interrogation can include device firmware revision information. In an example, the device data can include device parameters such as atrial or ventricular detection or therapy parameters. In an example, the device parameters can include one or more bradycardia parameters (such as upper and lower rate limit parameters, Atrial-to-Ventricular timing parameters, sensing configuration parameters, pacing output parameters, post-therapy brady parameters, etc.). In an example, the device parameters can include device mode information, battery status information, intrinsic amplitude measurements or alerts, lead impedance measurements, implantable medical device data/time information, encrypted patient identification data, change logs (for atrial & ventricular tachycardia and bradycardia modes), etc. In an example, device parameters can include episode summary (such as event ID, type, therapy summary, etc.), episode detail (such as event ID, type, detection and therapy details, etc.), EGM data, device marker data, etc.

In certain examples, IMD 120 is configured to receive an user input to initiate an initial interrogation of the IMED 120. In response, the external device 150 is configured to initiate transfer of first data (e.g., initial interrogation data) from the IMD 120 to the external device 150 during the initial interrogation. Also in response to the initiation of the initial interrogation, the external device 150 automatically initiates background transfer of second data (e.g., background interrogation data) from the IMD 120 to the external device 150 without requiring a user request for specifying the second data during the initial interrogation. In an example, the background transfer of second data (e.g., background interrogation data) is performed over a telemetry link 145. In an example, the background transfer of second data involves multiplexing the second data along with the first data and transmitting over the telemetry link 145. In an example, the background transfer of second data from the IMD 120 to the external device 150 begins before completing transfer of the first data from the MD 120 to the external device 150. In yet another example, the background transfer of second data from the IMD 120 to the external device 150 begins automatically after completing transfer of the first data from the IMD to the external device. In an example, the second data includes stored detail episode data, which can include historical physiological episode data.

In an example, the external device 150 can be configured to receive a user input requesting specific historical physiological episode data from the IMD. In response to the user input requesting specific historical physiological episode data, the automatically initiated background transfer of the second data is interrupted.

In an example, in response to the user input requesting the specific historical physiological episode data, the external device 150 is configured to preempt the automatically initiated retrieval of second data from the IMD, and to retrieve the specific historical physiological episode data from the IMD.

In an example, the external device 150 is configured to initiate transfer of first data from the IMD to the external device 150, the first data including at least one of device calibration data, device configuration data, device firmware data, device mode data, fault information data, magnet data, battery status data, episode identification data, impedance measurement data, encrypted patient data, trended data, atrial counter data, ventricular counter data, paced episode data or IMD date/time information.

In an example, the external device 150 is configured to initiate transfer the first data, which can include a plurality of operational parameter values used to control operation of the implantable medical device.

In an example, the external device 150 is configured to automatically initiate background transfer of second data during display of first data at the external device. In an example, the second data includes at least one of episode identification data, episode type data, episode detection data, therapy data, electrogram data, or device marker data.

In an example, the external device 150 is configured to transfer in the background the most recent historical physiological episode data in the implantable medical device followed then by background transfer of sequentially preceding historical physiological episode data during display of first data at the external device.

FIG. 1B is an illustration of an example of portions of a CRM system 100 and portions of the environment in which system 100 is used. In this example, the CRM system 100 includes an implantable system 115, an external system 155, and a telemetry link 145 providing bidirectional communication between the implantable system 115 and the external system 155. In this example, the implantable system 115 includes an implantable medical device 120 and a lead system 108. The implantable medical device 120 can be implanted within a body 102 and coupled to a heart 105 via lead system 108. Examples of implantable medical device 120 include, but are not limited to, pacemakers, pacemaker/defibrillators, cardiac resynchronization devices, cardiac remodeling control devices, and cardiac monitors. In certain examples, the lead system 108 can include multiple leads, such as including atrial or ventricular leads. In an example, the external system 155 can include a programmer. In another example, external system 155 can include a patient management system that can include an external device 150 in proximity of the implantable device 120, a remote device 170 that can be elsewhere, such as in a relatively distant location, and a telecommunication network 160 linking the external device 150 and the remote device 170. In this example, the patient management system allows access to the implantable system 115 from a remote location, such as for remotely monitoring patient status or adjusting one or more therapies. In an example, the telemetry link 145 provides data transmission from the implantable medical device 120 to the external system 155. This may include, for example, transmitting real-time physiological data acquired by the implantable medical device 120, extracting physiological data acquired by and stored in the implantable medical device 120, extracting therapy history data stored in the implantable medical device 120, or extracting data indicating an operational status of the implantable medical device 120 (e.g., battery status or lead impedance). In a further example, the telemetry link 145 provides data transmission from the external system 155 to the implantable medical device 120. This may include, for example, programming the implantable medical device 120 to acquire physiological data, programming the implantable medical device 120 to perform at least one self-diagnostic test (such as for a device operational status), or programming the implantable medical device 120 to deliver at least one therapy. In an example, the remote device 170 can be configured to receive from the external device 150 the second data transferred in the background upon initial interrogation of the IMD 120.

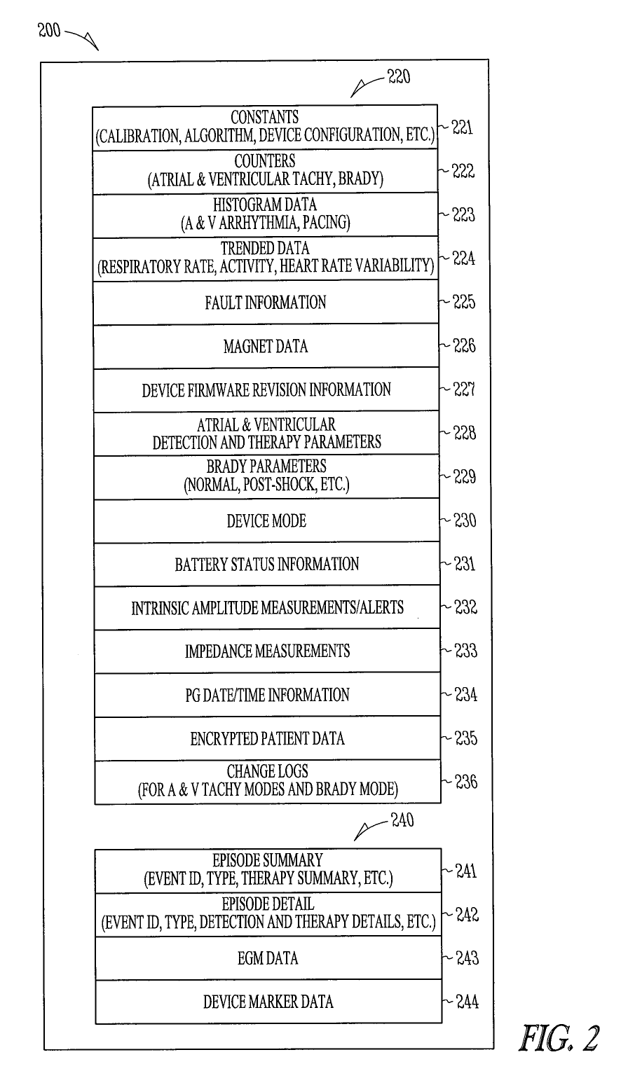

FIG. 2 is a block diagram illustrating an example of at least one kind of stored data 200 that can be transferred from the IMD to the external device during the interrogation of implantable medical devices. In an example, the stored data 200 includes the first data (or initial interrogation data) 220 and the second data (or background interrogation data) 240.

In an example, the initial interrogation data 220 includes at least one of calibration data, algorithm data, device configuration data 221, counter data (e.g., atrial or ventricular tachycardia counters, atrial or ventricular bradycardia counters) 222, histogram data (e.g., atrial or ventricular arrhythmia, pacing) 223, trended data (e.g., respiratory rate, activity, heart rate variability) 224, fault information 225, magnet data 226 which can configure the device behavior upon application of a magnetic field on the IMD. In some examples, magnet data 226 can be configured to initiate or withhold therapy when a magnetic field is applied on the IMD, device firmware revision information 227, atrial or ventricular detection or therapy parameters 228, bradycardia parameters (e.g., normal, post-shock, etc.) 229, device mode 230, battery status information 231, intrinsic amplitude measurements or alerts 232, lead impedance measurements 233, IMD (e.g., pulse generator) date or time information 234, encrypted patient data 235, change logs (e.g., for atrial or ventricular tachyarrhythmia modes or bradyarrhythmia modes) 236.

In an example, the background interrogation data 240 can include at least one of episode summary (e.g., event ID, type, therapy summary, etc.) 241, episode detail (e.g., event ID, type, detection or therapy details, etc.) 242, EGM (electrogram) data 243, or event marker data 244.

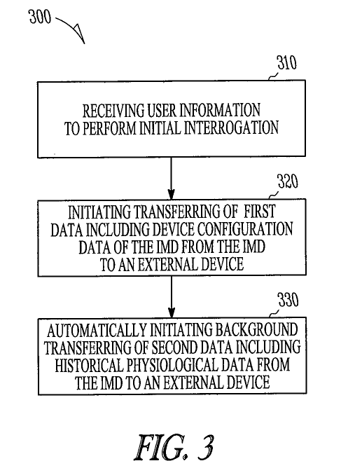

FIG. 3 illustrates an example of a method 300 for interrogating an implantable medical device (IMD) for a patient.

At 310, this includes receiving at an external device user input initiating an initial interrogation of an implantable medical device (IMD) using the external device.

At 320, transfer of first data from the IMD to the external device is initiated during the initial interrogation. In an example, the first data is displayed at the external device during the transfer of first data from the IMD to the external device. In an example, the first data includes device configuration data of the IMD.

At 330, background transfer of second data from the IMD to the external device is initiated during the initial interrogation and during display of first data at the external device, without requiring a specific user request for the second data. In an example, the background transfer of second data at 330 includes background transfer of historical physiological episode data. In an example, the background transfer of second data at 330 includes beginning transfer of second data from the IMD to the external device before completing transfer of the first data from the IMD to the external device. In an example, the background transferring of second data at 330 includes beginning transfer of second data from the IMD to the external device after completing transfer of the first data from the IMD to the external device.

In an example, method 300 can also include receiving, at the external device, user input requesting specific historical physiological episode data from the IMD. In an example, in response to the user input requesting specific historical physiological episode data, the automatically initiated background transfer of the second data from the IMD is interrupted. Then, the specific historical physiological episode data from the IMD is retrieved. In an example, method 300 includes resuming the automatically initiated background transferring of the second data after retrieving the specific historical physiological episode data from the IMD.

In an example, method 300 includes initiating transferring of first data, such as a plurality of operational parameter values used to control operation of the implantable medical device. In an example, method 300 includes transferring of first data such as at least one of device calibration data 221, device configuration data 221, device firmware data 227, device mode data 230, fault information data 225, magnet data 226, battery status data 231, impedance measurement data 233, encrypted patient data 235, trended data 224, atrial counter data 222, ventricular counter data 222, change logs 236 or MD date/time information 234.

In an example, method 300 includes transferring second data, such as retrieving stored detail episode information including at least one of an episode identification data, an episode type data, an episode detection data, a therapy data, a electrogram data and a device marker data.

In an example, method 300 includes background transferring of second data such as background transferring the most recent historical physiological episode data stored in IMD 120 followed then by background transferring sequentially preceding historical physiological episode data.

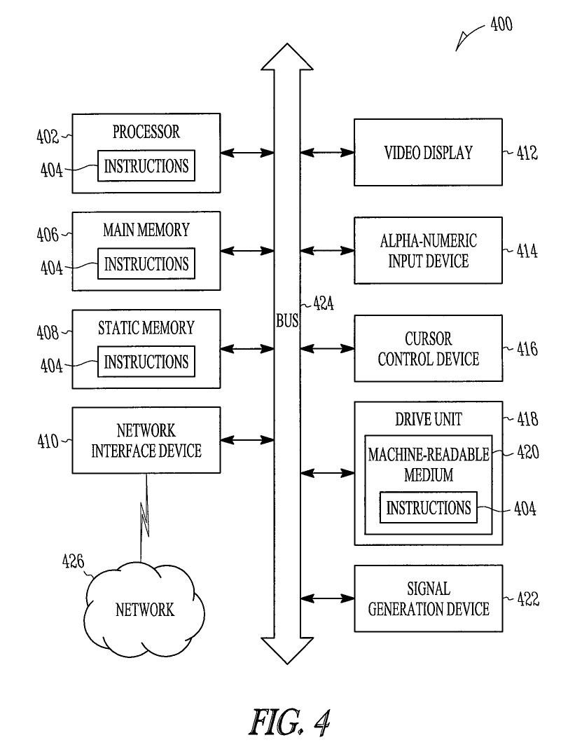

FIG. 4 illustrates a diagrammatic representation of a machine, such as a computer system 400. Instructions cause the machine to perform one or more of the techniques described herein. In certain examples, the machine can comprise a PRM, a computer, a network router, a network switch, a network bridge, Personal Digital Assistant (PDA), a cellular telephone, a web appliance, set-top box (STB) or any machine capable of performing instructions that specify actions to be taken by that machine. In various examples, the machine is configured to receiving a machine-readable medium that is encoded with instructions, wherein the instructions when executed comprises any of the various method steps described above for interrogating an implantable medical device (IMD) from an external device.

In the example of FIG. 4, the computer system 400 includes a processor 402, a main memory 406 and a static memory 408, which communicate with each other via a bus 424. In certain examples, the computer system 400 can further include a video display unit 412 (e.g., a liquid crystal display (LCD) or a cathode ray tube (CRT)). In certain examples, the computer system 400 also includes an alphanumeric input device 414 (e.g., a keyboard), a cursor control device 416 (e.g., a mouse), a disk drive unit 418, a signal generation device 422 (e.g., a speaker) and a network interface device 410 to interface the computer system to a network 426.

In certain examples the disk drive unit 418 includes a machine-readable medium 420 on which is stored a set of instructions or software 404 for performing one or more of the methodologies described herein. The software 404 is also shown to reside, completely or at least partially, within the main memory 406 or within the processor 402. The software 404 may further be transmitted or received via the network interface device 410. While the software is shown in FIG. 4 to reside within a single device, it will be appreciated that the software could be distributed across multiple machines or storage media, which may include the machine-readable medium.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventors also contemplate examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. A system comprising:
an implantable medical device (IMD); and
an external device in communication with the IMD and configured to receive an user input to perform an initial interrogation of the IMD using the external device, initiate transfer of first data from the IMD to the external device during the initial interrogation, display first data at the external device and automatically initiate background transfer of second data from the IMD to the external device during or after display of first data at the external device and without requiring a user request for the second data during the initial interrogation.

2. The system of claim 1, wherein the system is configured such that the background transfer of second data from the IMD to the external device begins before completing transfer of the first data from the IMD to the external device.

3. The system of claim 1, wherein the system is configured such that the background transfer of second data from the IMD to the external device begins after completing transfer of the first data from the IMD to the external device.

4. The system of claim 1, wherein the first data comprises at least one of device calibration data, device configuration data, device firmware data, device mode data, fault information data, magnet data, battery status data, impedance measurement data, encrypted patient data, trended data, atrial counter data, ventricular counter data, paced episode data or IMD date or time information.

5. The system of claim 1, wherein the second data comprises a stored detail episode data, the stored detailed episode data including historical physiological episode data corresponding to a particular episode.

6. The system of claim 1, wherein the external device is configured to receive a user input requesting specific historical physiological episode data from the IMD and in response to the user input requesting specific historical physiological episode data, to interrupt the automatically initiated background transfer of the second data.

7. The system of claim 6, wherein the external device is configured to preempt the automatically initiated background transfer of the second data from the IMD, and retrieve the specific historical physiological episode data from the IMD in response to the user input requesting the specific historical physiological episode data.

8. The system of claim 7, wherein the external device is configured to resume the automatically initiated background transfer of the second data from the IMD after retrieving the specific historical physiological episode data from the IMD.

9. The system of claim 1, wherein the external device is configured to initiate transfer of first data from the IMD to the external device, the first data including at least one of device calibration data, device configuration data, device firmware data, device mode data, fault information data, magnet data, battery status data, episode identification data, impedance measurement data, encrypted patient data, trended data, atrial counter data, ventricular counter data, paced episode data or IMD date or time information.

10. The system of claim 1, wherein the external device is configured to automatically initiate background transfer of second data including at least one of episode identification data, episode type data, episode detection data, therapy data, electrogram data, or device marker data.

11. The system of claim 1, wherein the external device is configured to transfer the first data including a plurality of operational parameter values used to control operation of the implantable medical device.

12. The system of claim 1 wherein the external device is configured to perform the background transfer of the second data to first transfer the most recent historical physiological episode data in the implantable medical device followed then by background transfer of preceding historical physiological episode data.

13. A method comprising:
receiving user input to perform an initial interrogation of an implantable medical device (IMD) using an external device, the initial interrogation commencing a communication session between the IMD and the external device;
during the initial interrogation, initiating transferring of first data from the IMD to the external device and displaying first data at the external device, wherein the first data comprises device configuration data of the IMD; and
during the initial interrogation, automatically initiating background transferring of second data from the IMD to the external device during or after display of first data at the external device, without requiring a user request for the second data, wherein the second data comprises historical physiological episode data.

14. The method of claim 13, wherein background transferring of second data includes beginning transfer of second data from the IMD to the external device before completing transfer of the first data from the IMD to the external device.

15. The method of claim 13, wherein background transferring of second data includes beginning transfer of second data from the IMD to the external device after completing transfer of the first data from the IMD to the external device.

16. The method of claim 13, comprising:
receiving, at the external device, user input requesting specific historical physiological episode data from the IMD; and
in response to the user input requesting specific historical physiological episode data, interrupting the automatically initiated background transfer of the second data from the IMD, and retrieving the specific historical physiological episode data from the IMD.

17. The method of claim 16, comprising:
resuming the automatically initiated background transferring of the second data after retrieving the specific historical physiological episode data from the IMD.

18. The method of claim 13, wherein transferring of first data includes transferring of first data including a plurality of operational parameter values used to control operation of the implantable medical device.

19. The method of claim 13, wherein transferring of first data includes transferring at least one of:
device calibration data, device configuration data, device firmware data, device mode data, fault information data, magnet data, battery status data, impedance measurement data, encrypted patient data, trended data, atrial counter data, ventricular counter data, paced episode data or IMD date or time information.

20. The method of claim 13, wherein the background transferring of second data comprises background transferring of stored detail episode information including at least one of episode identification data, episode type data, episode detection data, therapy data, electrogram data, or device marker data.

21. The method of claim 13, wherein the background transferring of second data comprises first background transferring most recent historical physiological episode data in the implantable medical device followed then by background transferring preceding historical physiological episode data.

22. A computer readable medium encoded with instructions, wherein the instructions when executed comprises:
receiving user input to perform an initial interrogation of the IMD using an external device, the initial interrogation commencing a communication session between the IMD and the external device;
during the initial interrogation, initiating transfer of first data from the IMD to the external device and displaying first data at the external device; and
during the initial interrogation, automatically initiating background transfer of second data from the IMD to the external device during or after display of first data at the external device, without requiring a user request for the historical physiological episode data, wherein the second data comprises historical physiological episode data.

23. The computer readable medium of claim 22, wherein the instructions when executed comprises:
beginning transfer of second data from the IMD to the external device before completing transfer of the first data from the IMD to the external device.

24. The computer readable medium of claim 22, wherein the instructions when executed comprises:
beginning transfer of second data from the IMD to the external device after completing transfer of the first data from the IMD to the external device.

25. The computer readable medium in claim 23, wherein the instructions when executed comprises:
receiving, at the external device, user input requesting specific historical physiological episode data from the IMD; and
in response to the user input requesting specific historical physiological episode data, preempting the automatically initiated background transferring of the second data comprising historical physiological episode data from the IMD, and retrieving the specific historical physiological episode data from the IMD.

26. The computer readable medium in claim 24, wherein the instructions when executed comprises:
resuming the automatically initiated background transferring of the second data after retrieving the specific historical physiological episode data from the IMD.
